(19) United States
(12) Patent Application Publication
(10) Pub. No.: US 2020/0098470 A1
(43) Pub. Date: Mar. 26, 2020

(54) TERMINAL DEVICE, INFORMATION PROCESSING SYSTEM, AND METHOD

(71) Applicant: SHARP KABUSHIKI KAISHA, Osaka (JP)

(72) Inventors: TOSHIROH MUKAI, Osaka (JP); MIHO HORII, Osaka (JP); MOTOKI YOSHIDA, Osaka (JP)

(21) Appl. No.: 16/577,626

(22) Filed: Sep. 20, 2019

(30) Foreign Application Priority Data
Sep. 25, 2018 (JP) 2018-179161

(51) Int. Cl. G16H 40/40; G16H 10/60; G06F 21/62

(52) U.S. Cl. CPC G16H 40/40; G06F 21/6245; G16H 10/60

(57) ABSTRACT

Public recommendation information that is obtained without referencing private information of an individual or a group is obtained, the public recommendation information is output for display, and private recommendation information that is obtained by referencing private information of an individual or a group is further output at a certain timing.

### BACKGROUND

### 1. Field

The present disclosure relates to a terminal device, an information processing system, and a method.

### 2. Description of the Related Art

There is a known schedule management technology where a user receives recommendations of tasks to be performed during vacant time in the user's schedule, in accordance with the circumstances of the user (Japanese Unexamined Patent Application Publication No. 2006-155368, published Jun. 15, 2006).

On the other hand, there has been a desire for users or groups including users to check public recommendation information and private recommendation information without troublesome operations. However, in order to reference information on behalf of a user regarding services of different service providers, troublesome procedures have to be performed such as having the user to register IDs and passwords, and furthermore, there has been a security issue regarding permission to use sensitive information such as IDs and the like.

It has been found desirable to realize a technology where public recommendation information and private recommendation information are presented to the user without having to perform troublesome operations.

### SUMMARY

A terminal device according to an aspect of the disclosure includes a communication unit and a control unit. The control unit is configured to obtain, via the communication unit, public recommendation information that is obtained without referencing private information of an individual or a group, output the public recommendation information, for display, and further output private recommendation information that is obtained by referencing private information of an individual or a group, at a certain timing.

### Advantageous Effects of Invention

An aspect of the disclosure advantageously provides the user with public recommendation information and private recommendation information, without having to perform troublesome operations.

### BRIEF DESCRIPTION OF THE DRAWINGS

FIG. 1 is a block diagram illustrating a schematic configuration of a television receiver, information processing device, and schedule server, according to an embodiment of the present disclosure;

FIG. 2 is a diagram schematically illustrating the configuration of the television receiver;

FIG. 3 is a sequence diagram illustrating the flow of information among the information processing device, television receiver, and schedule server;

Portions (a) and (b) of FIG. 4 are diagrams illustrating display examples of information, where (a) of FIG. 4 illustrates a display example of public recommendation information, and (b) of FIG. 4 illustrates a display example of private recommendation information;

FIG. 5 is a diagram illustrating an example of a public card;

FIG. 6 is a diagram illustrating an example of a private card; and

FIG. 7 a block diagram exemplifying the configuration of a computer that can be used as the information processing device and schedule server.

### DESCRIPTION OF THE EMBODIMENTS

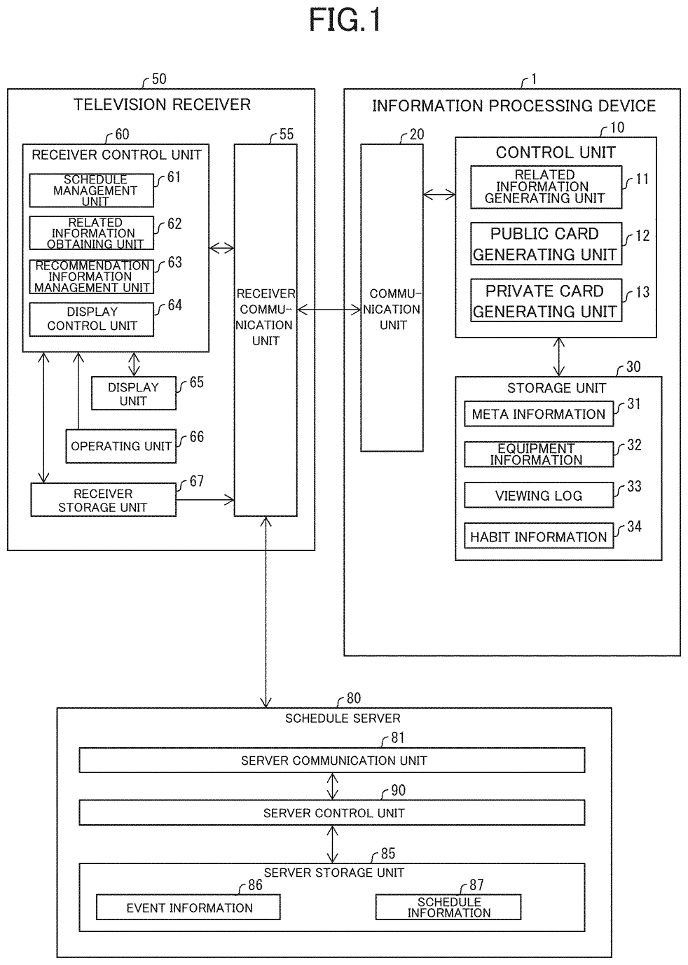

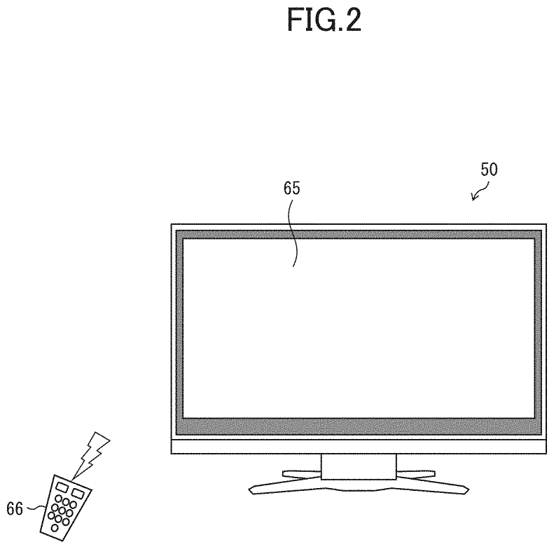

Embodiments of the present disclosure will be described below in detail. FIG. 1 is a block diagram illustrating a schematic configuration of a television receiver (terminal device) 50, an information processing device 1 to be connected to the television receiver 50, and a schedule server 80, according to an embodiment of the present disclosure. FIG. 2 is a diagram schematically illustrating the configuration of the television receiver 50.

### Configuration of Television Receiver 50

The television receiver 50 is provided with a receiver communication unit 55, a receiver control unit 60, a display unit 65, an operating unit 66, and a receiver storage unit 67, as illustrated in FIGS. 1 and 2. The television receiver 50 has a tuner that is omitted from illustration, and displays images corresponding to data streams received via the tuner on the display unit 65. Examples of the display unit 65 include display devices such as liquid crystal displays, organic electroluminescent (EL) displays (organic light-emitting displays (OLED)), plasma displays, and so forth.

Although the television receiver 50 having a tuner that receives broadcast waves is described as an example of a terminal device in the present embodiment, a configuration of the present embodiment may be realized by connecting a terminal device such as a display device or the like that does not have a tuner to the information processing device 1 and schedule server 80. The terminal device according to the present embodiment may also be a mobile terminal device, such as a tablet personal computer (PC) or smartphone.

The operating unit 66 may be operating buttons (not shown) or the like that are integrally provided to the television receiver 50, or may be a remote controller, smartphone, or the like, that is separate from the television receiver 50.

The receiver storage unit 67 is storage that stores various types of data to be used by the receiver control unit 60. The receiver storage unit 67 can be realized by one of, or a combination or one or more of erasable programmable read-only memory (EPROM), electrically erasable programmable read-only memory (EEPROM, a registered trademark), a hard disk drive (HDD), flash memory, and so forth, which are non-volatile memory of which the contents can be rewritten, for example.

The receiver communication unit 55 is configured to be capable of connecting to a network such as the Internet or the like by wireless communication or wired communication, and is connected to the information processing device 1 and schedule server 80 via a network. The receiver communication unit 55 functions as a transmission unit that externally transmits regional information that the television receiver 50 has.

The receiver control unit 60 is a computing unit that has functions of centrally controlling each part of the television receiver 50.

### Functional Blocks of Receiver Control Unit 60

The receiver control unit 60 has a schedule management unit 61, a related information obtaining unit 62, a recommendation information management unit 63, and a display control unit 64.

### Schedule Management Unit

The schedule management unit 61 is realized, for example, by the television receiver 50 executing a schedule management application for data sharing with the schedule server 80. The schedule management unit 61 accepts user instructions relating to a schedule, and stores private schedule information relating to a schedule of the user (individual) or a group including the user in the receiver storage unit 67. The schedule management unit 61 also transmits the private schedule information to the schedule server 80, and the schedule server 80 obtains and manages the private schedule information.

Examples of private schedule information here include
information indicating that certain private event information has been registered in a schedule,
information indicating a schedule (date and time, and duration) of certain private event information,
information indicating a location (region, geographical name, name of a spot, or latitude/longitude) correlated with certain private event information,
information indicating another user or a group including the user correlated with certain private event information, and
   examples of private event information include
birthday of a user or a member of a group including the user,
entrance or graduation ceremony of a user or a member of a group including the user,
work plan of a user or a member of a group including the user,
plan of a user or a member of a group including the user to visit a certain place,
plan of a user or a member of a group including the user to meet another user, and so forth,
   but are not restricted to these.

Note that the schedule server 80 manages individual private schedule information for each of a large number of schedule usage IDs, and obtains schedule information, correlated with a schedule usage ID input to the aforementioned schedule management application, from the schedule management unit 61. Accordingly, an example of the aforementioned “private schedule information relating to a schedule of the user or a group including the user” is “private schedule information correlated with a schedule usage ID usable by the user or a group including the user”.

Note that a group including the user means a group made up of members using the same schedule usage ID as the user, such as a family to which the user belongs, a group made up of the user and housemates thereof, or the like, for example.

The user may be able to transmit private schedule information to the schedule server 80 from a terminal device such as a PC or smartphone or the like, for example, using a common schedule usage ID, so as to be managed, although this is omitted from illustration. The schedule management unit 61 may obtain private schedule information correlated with a schedule usage ID usable by the user or a group including the user from the schedule server 80, and update the private schedule information stored in the receiver storage unit 67, by executing the schedule management application at predetermined periods.

### Related Information Obtaining Unit

The related information obtaining unit 62 is realized, for example, by the television receiver 50 executing an application for data sharing with the information processing device 1. The related information obtaining unit 62 externally obtains related information, which is information relating to event information, via the receiver communication unit 55, in accordance with the content of event information that the schedule management unit 61 has registered in a schedule, for example. The related information that has been obtained is stored in the receiver storage unit 67.

In the present embodiment, the related information obtaining unit 62 obtains event information from the schedule management unit 61. On the other hand, the related information obtaining unit 62 obtains related information from the information processing device 1. The related information obtaining unit 62 may extract and obtain related information from information stored in the receiver storage unit 67.

### Recommendation Information Management Unit

The recommendation information management unit 63 is realized, for example, by executing an application for managing recommendation information to be displayed on the display unit 65. The recommendation information management unit 63 obtains public recommendation information that can be obtained without referencing private information of a user or a group including the user, from the information processing device 1 via the receiver communication unit 55. An example of “private information” here is the aforementioned “private event information”.

The recommendation information management unit 63 may also obtain a group of data not including private recommendation information of a user or a group including the user and including period information indicating a display period for displaying the private recommendation information, from the information processing device 1 via the receiver communication unit 55. Examples of the aforementioned public recommendation information and the aforementioned group of data will be described later.

Also, the recommendation information management unit 63 may obtain private information of a user or a group including the user from an application functioning as the schedule management unit 61, and reference the obtained private information to generate private recommendation information of a user or a group including the user. The recommendation information management unit 63 may generate private recommendation information here by referencing related information relating to private event information, which the related information obtaining unit 62 has obtained.

Note that a configuration may be made where obtaining of information from the schedule management unit 61 by the recommendation information management unit 63 is realized by the application functioning as the recommendation information management unit 63 calling an application programming interface (API) of the application functioning as the schedule management unit 61.

### Display Control Unit

At least part of the functions of the display control unit 64 is realized, for example, by executing an application for controlling display content to be displayed on the display unit 65. The display control unit 64 causes public recommendation information obtained by the recommendation information management unit 63 to be displayed on the display unit 65. The display control unit 64 also is configured to be able to further display private recommendation information generated by the recommendation information management unit 63 on the display unit 65, at a certain timing.

The display control unit 64 may display private recommendation information at at least one timing of a display period specified by period information obtained by the recommendation information management unit 63. Specific examples of screens that the display control unit 64 displays on the display unit 65 will be described later.

According to these configurations, the user can be presented with public recommendation information that can be obtained without referencing private information of the user or a group including the user, and private recommendation information relating to private information of the user or a group including the user, without having to perform troublesome operations.

### Configuration of Information Processing Device 1

The information processing device 1 includes a communication unit 20, a control unit 10, and a storage unit 30. The communication unit 20 communicates with the television receiver 50 and schedule server 80 by wireless communication or wired communication. Although FIG. 1 illustrates one television receiver 50 only, the information processing device 1 is configured so as to be capable of communicating with one or multiple television receivers 50 via the communication unit 20.

The control unit 10 is a computing unit that has functions of centrally controlling each part of the information processing device 1. The control unit 10 has a related information generating unit 11, a public card generating unit 12, and a private card generating unit 13.

The related information generating unit 11 is realized, for example, by the information processing device 1 executing an application for data sharing with the television receiver 50. The related information generating unit 11 references information stored in the storage unit 30 and generates related information to be transmitted to a relevant television receiver 50, which will be described in detail later.

The related information generating unit 11 transmits related information that has been generated to the television receiver 50 via the communication unit 20. The related information generating unit 11 generates related information every day, at a predetermined time that has been set beforehand, and transmits the related information to the television receiver 50. The related information generating unit 11 may also be able to generate related information in response to a request from the television receiver 50, and transmit the related information to the television receiver 50. Alternatively, the related information generating unit 11 may be able to generate related information multiple times a day, and transmit the related information to the television receiver 50. An arrangement may also be made where the user is able to set the time of reception of related information from the information processing device 1 at the television receiver 50, by performing operations on the television receiver 50.

The public card generating unit 12 is realized, for example, by the information processing device 1 executing an application for data sharing with the television receiver 50. The public card generating unit 12 generates a public card including public recommendation information that can be obtained without referencing private schedule information of the user or a group including the user. Information included in a public card will be referred to as public data. Public data will be described later.

The public card generating unit 12 transmits the public card that has been generated to the television receiver 50 via the communication unit 20. The public card generating unit 12 generates a public card every day, at a predetermined time that has been set beforehand, and transmits the public card to the television receiver 50. The public card generating unit 12 may also be able to generate a public card in response to a request from the television receiver 50, and transmit the public card to the television receiver 50. Alternatively, the public card generating unit 12 may also be able to generate a public card multiple times a day, and transmit the public card to the television receiver 50. An arrangement may also be made where the user is able to set the time of reception of public cards from the information processing device 1 at the television receiver 50, by performing operations on the television receiver 50.

The private card generating unit 13 is realized, for example, by the information processing device 1 executing an application for data sharing with the television receiver 50. The private card generating unit 13 generates a private card not including private recommendation information of a user or a group including the user and including period information indicating a display period for displaying the private recommendation information. Information included in a private card will be referred to as private data (a group of data). Private data will be described later.

The private card generating unit 13 transmits the private card that has been generated to the television receiver 50 via the communication unit 20. The private card generating unit 13 generates a private card every day, at a predetermined time that has been set beforehand, and transmits the private card to the television receiver 50. The private card generating unit 13 may also be able to generate a private card in response to a request from the television receiver 50, and transmit the private card to the television receiver 50. Alternatively, the private card generating unit 13 may also be able to generate a private card multiple times a day, and transmit the private card to the television receiver 50. An arrangement may also be made where the user is able to set the time of reception of private cards from the information processing device 1 at the television receiver 50, by performing operations on the television receiver 50.

The storage unit 30 is storage that stores various types of data usable by the control unit 10. The storage unit 30 can be realized by one of, or a combination or one or more of EPROM, EEPROM (a registered trademark), HDD, flash memory, and so forth, which are non-volatile memory of which the contents can be rewritten, for example.

The storage unit 30 stores meta information 31, equipment information 32, viewing logs 33, and habit information 34. The meta information 31 is information relating to contents of television programs, and includes information of products, shops, and so forth, introduced in a program.

The equipment information 32 includes information relating to one or multiple television receivers 50 registered beforehand to receive information provided from the information processing device 1. The equipment information 32 includes a terminal ID unique to each television receiver 50, and a member ID of the user using the television receiver 50 having the terminal ID. The control unit 10 of the information processing device 1 identifies the television receiver 50 that is the object, using at least one of the terminal ID and the member ID of the user.

The equipment information 32 also includes region information indicating the location where each television receiver 50 is installed. The region information may be information such as the name of a municipality, zip code, address, or the like, for example. The equipment information 32 may also include profile information such as gender and age, interests, and so forth of the user.

When a television receiver 50 is being registered as equipment that is the object of receiving related information, the control unit 10 of the information processing device 1 obtains the terminal ID of the television receiver 50, member ID of the user, and equipment information such as region information where the television receiver 50 is installed and so forth, from the television receiver 50 via the communication unit 20. The control unit 10 of the information processing device 1 correlates equipment information obtained from television receivers 50 with respective terminal IDs, and stores the correlated equipment information in the storage unit 30 as equipment information 32.

The viewing log 33 is information relating to viewing history of the television receiver 50, obtained from the television receiver 50, and is stored correlated with the terminal ID of each television receiver 50.

The habit information 34 is information that can be obtained by referencing operation history of the television receiver 50, which is obtained from the television receiver 50, for example. The habit information 34 may include past viewing history of the user, and information regarding viewing habits of the user (habits regarding channels, genres, cast, and so forth) and viewing time-of-day (e.g., habits regarding viewing time-of-day for each day of the week), and so forth, that can be obtained based on usage history of the equipment, correlated with the member ID of the user. The habit information 34 may also include profile information such as gender and age, interests, and so forth of the user.

The related information generating unit 11 may also be able to generate related information to be transmitted to the television receiver 50 that is the object, with reference to the meta information 31, equipment information 32, viewing log 33, and habit information 34 stored in the storage unit 30. The following is lists of examples of information included in the meta information 31, equipment information 32, viewing log 33, and habit information 34.

### Meta Information 31

title of program
genre of program
cast of program
product(s) introduced in program
shops(s) introduced in program
region(s) introduced in program
product(s) introduced in commercials run during program

### Equipment Information 32

equipment ID
type of equipment
user ID of user of equipment
installation location of equipment (region where installed)

### Viewing Log 33

equipment ID
title of program
genre of program
date (calendar date, day of week, time of day)
broadcasting region

### Habit Information 34

user ID
viewing history
viewing habits (operating history and running history of television receiver 50)
date
interests
preferences
gender
age
   The related information generating unit 11 may also be able to generate program listings for each region, each genre, each cast, and so forth, by referencing the meta information 31.

The public card generating unit 12 may also generate a public card including information of television programs recommended for viewing to a user or a group including the user, by referencing the meta information 31 stored in the storage unit 30, region information of the television receiver 50, viewing history of the user or group including the user, information such as viewing habits of the user or group including the user, and so forth. The public card generating unit 12 may also generate multiple public cards including information indicating degree of priority (ranking of recommendations) for recommendation to the user or group including the user, in accordance with viewing history of the user or group including the user, information such as viewing habits of the user or group including the user, and so forth, stored in the storage unit 30.

The private card generating unit 13 may also generate a private card including period information indicating a display period for displaying private recommendation information, by referencing the meta information 31 stored in the storage unit 30, region information of the television receiver 50, viewing history of the user or group including the user, information such as viewing habits of the user or group including the user, and so forth.

### Configuration of Schedule Server 80

The schedule server 80 includes a server communication unit 81, a server control unit 90, and a server storage unit 85.

The server communication unit 81 communicates with the television receiver 50 by wireless communication or wired communication. The schedule server 80 obtains private schedule information of a user or members of a group including the user from the television receiver 50 via the server communication unit 81, and manages the information.

The server control unit 90 is a computing unit that has functions of centrally controlling each part of the schedule server 80.

The server storage unit 85 stores event information 86 and schedule information 87.

The event information 86 stores private event information of a user or members of a group including the user, correlated with the schedule usage ID. The event information 86 may also store public event information that is not correlated with the schedule usage ID. Public event information may include information relating to events such as festivals, galas, workshops, garbage truck schedules, sales at shops, and so forth, in correlation with information relating to the region, for example.

The schedule information 87 stores schedule information relating to private event information of a user or members of a group including the user, correlated with the schedule usage ID.

The schedule server 80 is a server operated by a service provider that provides a Web application for schedule management and so forth (calendar service). The schedule server 80 stores event information registered by the user by operating the television receiver 50, and schedule information related to the event information, in the server storage unit 85, each in correlation with the schedule usage ID of the user.

### Flow of Information

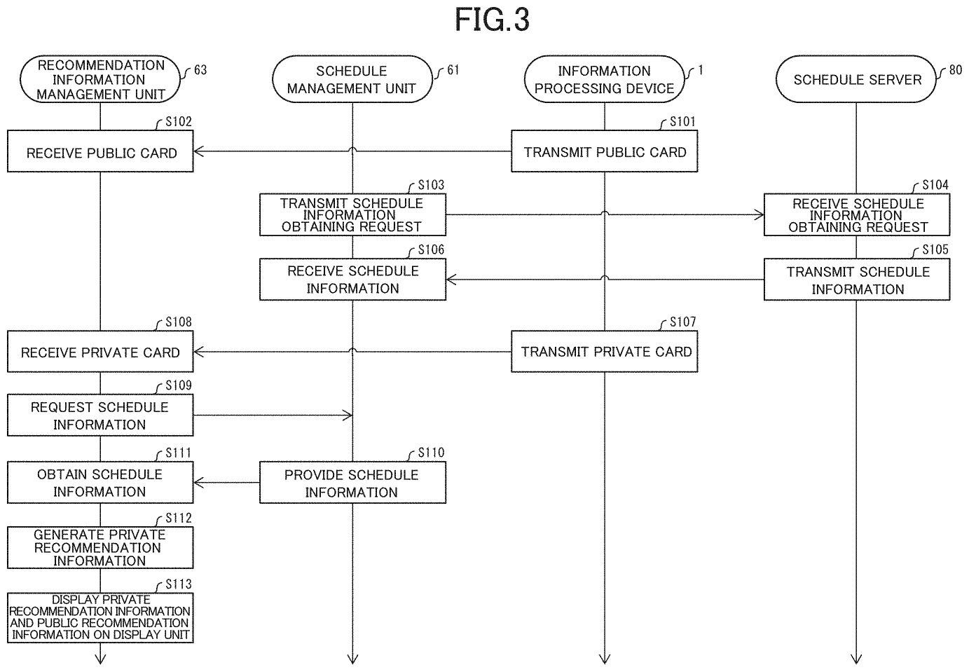

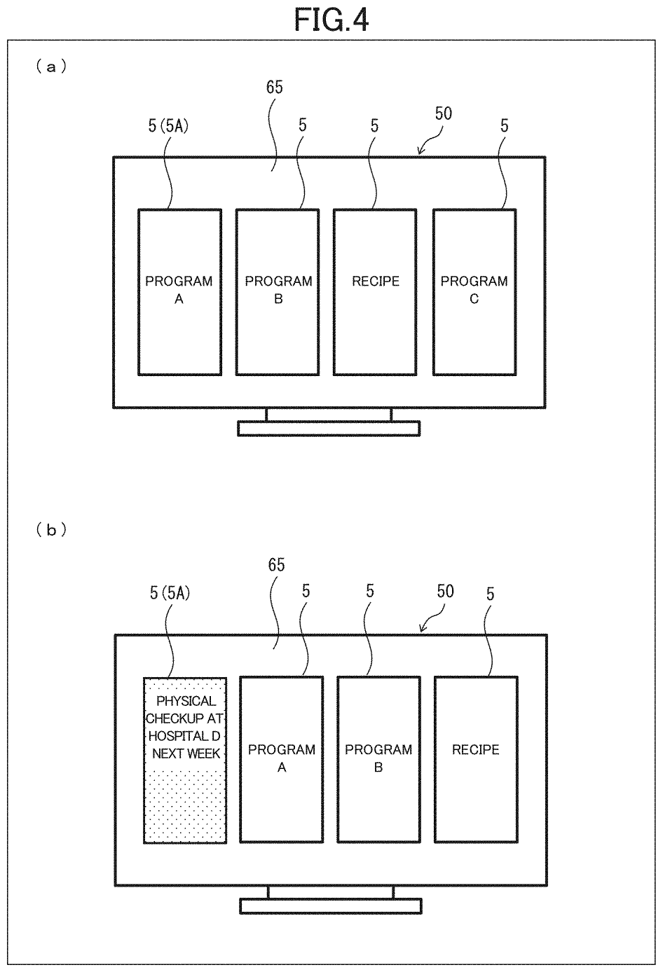

FIG. 3 is a sequence diagram illustrating the flow of information among the information processing device 1, recommendation information management unit 63, schedule management unit 61, and schedule server 80.

### Step S101

The control unit 10 of the information processing device 1 generates a public card using the functions of the public card generating unit 12, and the public card is then transmitted to the television receiver 50 via the communication unit 20. The control unit 10 of the information processing device 1 transmits multiple public cards to the television receiver 50 at a predetermined time that has been set beforehand.

### Step S102

The recommendation information management unit 63 receives a public card via the receiver communication unit 55.

### Step S103

The schedule management unit 61 transmits a schedule information obtaining request to the schedule server 80 at a predetermined timing.

### Step S104

The server control unit 90 of the schedule server 80 receives the schedule information obtaining request via the server communication unit 81.

### Step S105

The server control unit 90 of the schedule server 80 references the schedule ID included in the schedule information obtaining request that has been received, and transmits schedule information stored in the server storage unit 85.

### Step S106

The schedule management unit 61 receives schedule information via the receiver communication unit 55. The schedule management unit 61 synchronizes schedule information with the schedule server 80 by executing the processing of steps S103 through S106 at a predetermined timing.

### Step S107

The control unit 10 of the information processing device 1 generates a private card using the functions of the private card generating unit 13, and transmits the private card via the communication unit 20.

### Step S108

The recommendation information management unit 63 receives the private card via the receiver communication unit 55.

### Step S109

The recommendation information management unit 63 requests the schedule management unit 61 to provide schedule information relating to private event information of the user or members of the group including the user, based on conditions (period information indicating a display period for displaying the private recommendation information) within the private card.

### Step S110

The schedule management unit 61 provides the recommendation information management unit 63 with schedule information relating to private event information of the user or members of the group including the user.

### Step S111

The recommendation information management unit 63 obtains the schedule information relating to the private event information of the user or members of the group including the user.

### Step S112

The recommendation information management unit 63 supplements the private card including period information indicating a display period for displaying the private recommendation information with private schedule information obtained from the application functioning as the schedule management unit 61, and generates private recommendation information of a user or group including the user.

### Step S113

The recommendation information management unit 63 displays, on the display unit 65, public recommendation information based on the public card and private recommendation information generated by supplementing the private card with private schedule information.

### Examples of Display on Television Receiver 50

FIGS. 4A and 4B are diagrams illustrating display examples of recommendation information on the television receiver 50. The receiver control unit 60 of the television receiver 50 uses the functions of the display control unit 64 to display at least multiple cards including public recommendation information at a display position of the display unit 65 in accordance with a degree of priority correlated with each card, at a certain predetermined timing. The display control unit 64 also may display multiple cards including public recommendation information, and at least one card including private recommendation information, at a display position of the display unit 65 in accordance with a degree of priority correlated with each card, at a certain different predetermined timing. The display control unit 64 may display a card including private recommendation information at a display position with a higher ranking in priority than multiple cards including public recommendation information.

Portions (a) of FIG. 4 is a diagram illustrating a display example of recommendation information on the television receiver 50 in a case where there is no private recommendation information, or a case where it is not the timing to display private recommendation information. In the example illustrated in (a) of FIG. 4, four display positions for cards including recommendation information are set on the display unit 65, and are set such that out of these, the display position with the highest ranking in priority is at the left side of the screen. That is to say, the display control unit 64 displays, out of multiple cards including public recommendation information, four cards ranking high in priority, arrayed on the display unit 65 from the left in order of recommendation.

Cards 5 including public recommendation information are cards including information of programs currently being broadcast or to be broadcast in the future, generated based on, for example, viewing history of a user or members of a group including the user, and viewing habits. Cards including information of “program A”, “program B”, and “program C”, which are cards including information of these programs, are displayed on the display unit 65 at display positions corresponding to their degree of priority.

Also, a card 5 including public recommendation information may be a card including information relating to other electric equipment collaborating with the television receiver 50 through a system. Examples of other electric equipment collaborating with the television receiver 50 through a system include home appliances used in the building where the television receiver 50 is installed such as a house, which are home appliances such as electric cooking appliances, refrigerator, and so forth.

Equipment information, operating history, and running history of the other electric equipment may be stored in the information processing device 1 along with the equipment information, operating history, and running history of the television receiver 50, correlated by user ID. The control unit 10 of the information processing device 1 may be able to use the functions of the public card generating unit 12 to generate public cards including information relating to the other electric equipment. A “recipe” card including information of procedures for cooking using an electric cooking appliance collaborating with the television receiver 50 through a system, for example, may be displayed on the display unit 65 of the television receiver 50.

Also, the user can set beforehand contents of cards to display as recommendation information (e.g., weather forecast, top news, etc.), and cards corresponding to user settings may be displayed on the display unit 65 along with cards including event information.

Portions (b) of FIG. 4 is a diagram illustrating a display example of recommendation information on the television receiver 50 in a case where there is private recommendation information at the timing of displaying private recommendation information. In the example illustrated in (b) of FIG. 4, a card including private recommendation information is displayed at a display position 5A at the left side of the screen, which is the display position with the highest ranking in priority out of the four display positions for cards including recommendation information that have been set on the display unit 65. Note that in a case where there are multiple cards including private recommendation information, the display control unit 64 may display, in order in accordance with the degree in priority, multiple cards including private recommendation information at display positions with a higher ranking in priority than multiple cards including public recommendation information.

Cards including private recommendation information are cards including information relating to private schedule information of a user or a group including the user. For example, in a case where event information having date and time information that is called “physical checkup” is registered in the schedule of a user or a group including the user, private recommendation information “physical checkup at hospital D next week” is generated by the recommendation information management unit 63. The recommendation information management unit 63 generates recommendation information, using a private card including period information indicating a display period for display of private recommendation information, at a date and time prior to the date that the date and time information that the registered schedule information indicates, one week prior for example.

The display control unit 64 may display at least one card including private recommendation information generated by the recommendation information management unit 63 along with multiple cards including public recommendation information, at display positions corresponding to their respective rankings in priority, at a timing corresponding to period information indicating a display period included in private cards.

The display control unit 64 displays the card stating, “physical checkup at hospital D next week” including private recommendation information at a display position having a higher ranking in priority than the cards for “program A”, “program B”, and “recipe” that include public recommendation information.

Note that an arrangement may be made where the user can optionally set the timing of displaying cards including public recommendation information and the timing of displaying cards including private recommendation information. For example, the user may be able to perform settings to display cards including private recommendation information a predetermined number of days, such as three days, before the day that the date and time information of the schedule information indicates, on the display unit 65 of the television receiver 50. Also, the user may be able to perform settings to display cards including private recommendation information relating to plans for the weekend next week on the display unit 65 of the television receiver 50, on the weekend of the preceding week. Also, the user may be able to perform settings to display cards including private recommendation information relating to plans for the coming week starting from tomorrow, on the display unit 65 of the television receiver 50.

The control unit 10 of the information processing device 1 may also use the functions of the private card generating unit 13 to reference user viewing history and user viewing habits to generate private cards including period information indicating a display period of displaying private recommendation information, in accordance with a date and time where the probability of cards including private recommendation information will be seen by the user is high.

Now, there are cases where cards including private recommendation information are not displayed at the date and time corresponding to the period information indicating the display period for displaying cards including private recommendation information, included in private cards, due to the user being away from home or the like and the power of the television receiver 50 not being turned on. In such a case, the display control unit 64 of the television receiver 50 may be able to display cards including private recommendation information on the display unit 65 at a later time, at the timing of the power of the television receiver 50 being turned on.

Note that private recommendation information is not restricted to information relating to event information that the schedule management unit 61 has registered in a schedule, and may include information obtained from information obtained by the related information obtaining unit 62, in accordance with the contents of event information that the schedule management unit 61 has registered in a schedule. For example, in a case where event information having date and time information, such as plans for a business trip or an outing or the like including information regarding a destination, has been registered in a schedule of a user or a group including the user, the recommendation information management unit 63 may reference the related information obtained by the related information obtaining unit 62, and generate private recommendation information including information of programs relating to the destination registered in the schedule. Cards including private recommendation information indicating information selected from related information by referencing private schedule information are displayed at display positions with a higher ranking in priority than cards including public recommendation information.

### Public Cards

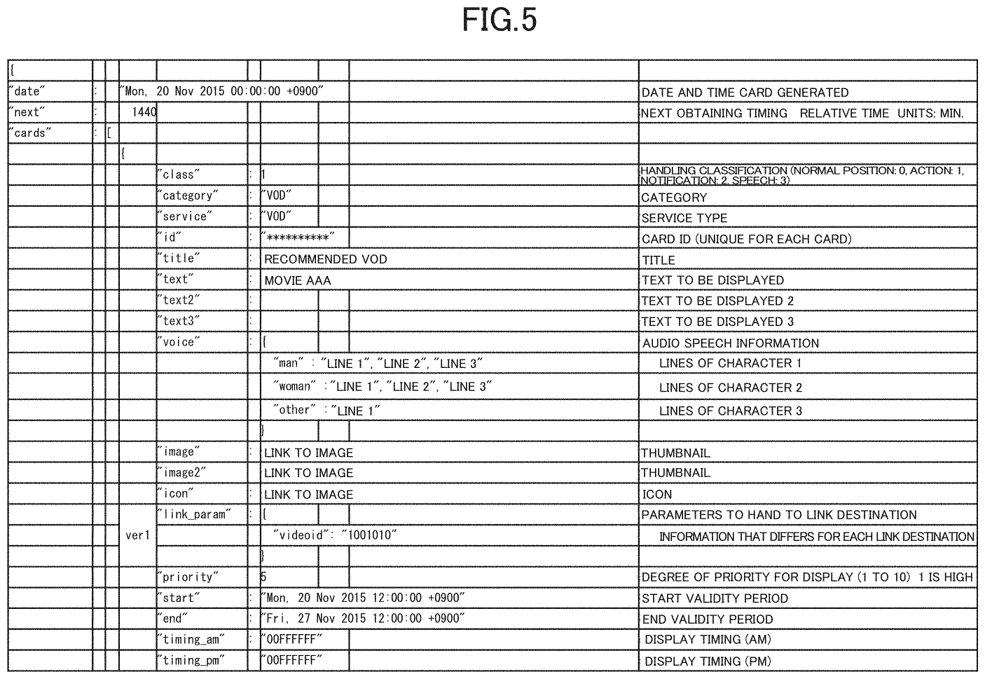

FIG. 5 is a diagram illustrating an example of a public card that the control unit 10 of the information processing device 1 generates using the functions of the public card generating unit 12. A public card includes public recommendation information that can be obtained without referencing information of an individual or group, as illustrated in FIG. 5. A public card includes information relating to
date and time when the card was generated,
timing of the card being obtained next by the television receiver 50,
type of card (handling classification, category, service type, ID, etc.),
contents of recommendation information (title, texts to be displayed, audio, images, etc.),
degree of priority for display,
period of validity, and
display timing,
   as illustrated in FIG. 5.

The type of card includes information indicating the category of information, such as whether the recommendation information is a television program, video on demand (VOD), or the like. Contents of the recommendation information include the title of the program or VOD, texts indicating recommendation information to be displayed on the display unit 65 of the television receiver 50, audio data indicating recommendation information for audio output from the television receiver 50 (e.g., lines spoken by characters, etc.), thumbnails of images representing the recommendation information, and so forth.

Degree of priority of display is represented on a scale of 1 to 10, with 1 being recommendation information with the highest degree of priority, and 10 being recommendation information with the lowest degree of priority, for example. The period of validity includes information relating to a period of validity during which public recommendation information based on the public card can be displayed on the display unit 65 of the television receiver 50, and a period of several days, for example, may be specified. The display timing includes information regarding at which timing of the day that public recommendation information based on the public card can be displayed on the display unit 65 of the television receiver 50 during the aforementioned period of validity. The display timing may be set to multiple times per day, such as a such-and-such time AM and a such-and-such time PM, for example.

### Private Cards

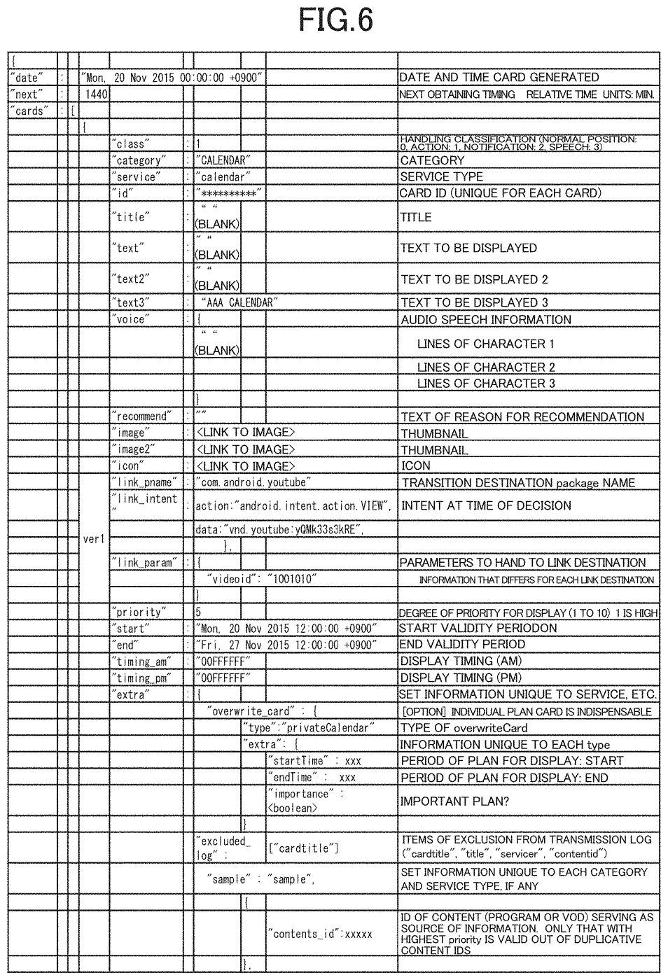

FIG. 6 is a diagram illustrating an example of a private card that the control unit 10 of the information processing device 1 generates using the functions of the private card generating unit 13. A private card does not include private recommendation information that is not obtainable without referencing information of an individual or a group, but rather includes period information indicating a display period for displaying private recommendation information, as illustrated in FIG. 6. A private card includes information relating to
date and time when the card was generated,
timing of the card being obtained next by the television receiver 50,
type of card (handling classification, category, service type, ID, etc.),
degree of priority for display,
period of validity,
period of display, and
display timing,
   as illustrated in FIG. 6.

The type of card includes information indicating that the recommendation information is information that can be obtained by referencing private schedule information of the individual user or group including the user.

Degree of priority of display is represented on a scale of 1 to 10, with 1 being recommendation information with the highest degree of priority, and 10 being recommendation information with the lowest degree of priority, for example. The period of validity includes information relating to a period of validity during which private recommendation information based on the private card can be displayed on the display unit 65 of the television receiver 50, and a period of several days, for example, may be specified. The display timing includes information regarding at which timing of the day that private recommendation information based on the private card can be displayed on the display unit 65 of the television receiver 50 during the aforementioned period of validity. The display timing may be set to multiple times per day, such as a such-and-such time AM and a such-and-such time PM, for example.

A private card includes fields for the title, texts to be displayed, audio, images, and so forth, where the contents of the recommendation information are to be filled in. These fields where the contents of the recommendation information are to be filled in are blank at the time of generating the private card. These blank portions are supplemented by the recommendation information management unit 63 of the television receiver 50 referencing schedule information of the user or members of group including the user, obtained by the schedule management unit 61.

Further, a private card includes period information relating a display period of display of private recommendation information based on the private card on the display unit 65 of the television receiver 50. The period information includes a display start scheduled time of the private recommendation information, based on the private card, on the display unit 65 of the television receiver 50, and a display end scheduled time.

A private card also includes fields where information indicating the degree of importance of the schedule is to be filled in, which is supplemented by the recommendation information management unit 63. The display control unit 64 may decide the ranking of priority of multiple cards including private recommendation information, by referencing information indicating the degree of importance of a schedule in private recommendation information that has been generated by the recommendation information management unit 63 supplementing information of the private card.

Also, a private card has described therein to enable only recommendation information with the highest degree of priority in a case where the private recommendation information generated by the recommendation information management unit 63 is a duplication of public recommendation information.

### Supplemental Items

Note that a configuration has been described in the present embodiment where a terminal device, of which the television receiver 50 is an example, communicates with the schedule server 80, and displays private recommendation information that can be obtained by referencing individual or group information stored in the schedule server 80 on the display unit 65. The terminal device, of which the television receiver 50 is an example, is not restricted to communication with the schedule server 80, and can communicate with a VOD server or the like for example, and display private recommendation information obtained by referencing individual or group information stored in such servers on the display unit 65. Information of an individual or group stored in a VOD server may be information relating to VOD viewing history of the individual or group, for example, and the terminal device may be able to display information relating to private recommendation videos obtained by referencing individual or group viewing history on the display unit 65.

### Embodiment 2

Although an example of using one each of the information processing device 1 and schedule server 80 has been described in the above embodiment, the functions that the information processing device 1 and schedule server 80 have may each be realized in separate servers. In a case of applying multiple servers, each server may be managed by the same service provider, or may be managed by different service providers.

### Embodiment 3

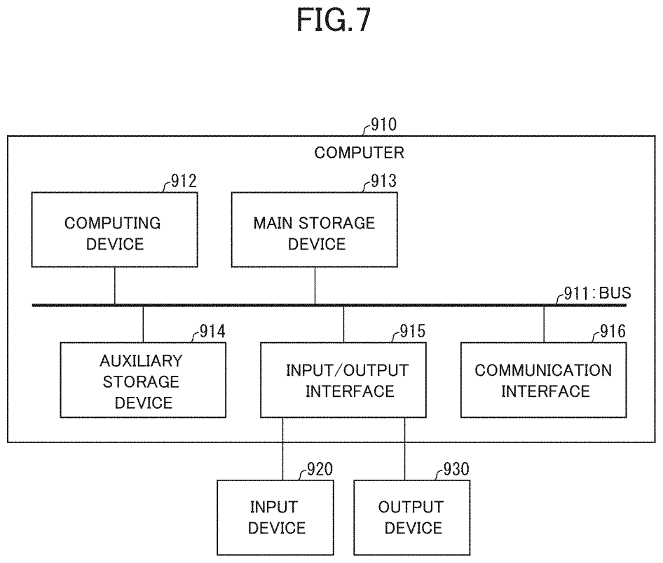

The blocks of the information processing device 1 and schedule server 80 may be realized by logical circuits (hardware) formed of integrated circuit (IC) chips or the like, or may be realized by software. In the case of the latter, the information processing device 1 and schedule server 80 may each be configured using a computer such as illustrated in FIG. 7.

FIG. 7 is a block diagram exemplifying the configuration of a computer 910 that can be used as the information processing device 1 and schedule server 80. The computer 910 has a computing device 912, a main storage device 913, an auxiliary storage device 914, an input/output interface 915, and a communication interface 916, which are mutually connected via a bus 911. The computing device 912, main storage device 913, and auxiliary storage device 914 may each be, for example, one processor (e.g., a central processing unit (CPU) or the like) or more, one random access memory (RAM) or more, and one hard disk drive or more. An input device 920 for the user to input various types of information to the computer 910, and an output device 930 for the computer 910 to output various types of information to the user, are connected to the input/output interface 915. The input device 920 and output device 930 may be built into the computer 910, or may be connected (externally attached) to the computer 910. For example, the input device 920 may be a keyboard, mouse, touch sensor, or the like, and the output device 930 may be a display, printer, speaker, or the like. Also, a device having the functions of both the input device 920 and output device 930, like a touch panel where a touch sensor and display have been integrated, may be applied. The communication interface 916 is an interface for the computer 910 to communicate with external devices.

Various types of programs for causing the computer 910 to operate as the information processing device 1 and schedule server 80 are stored in the auxiliary storage device 914. The computing device 912 loads the programs stored in the auxiliary storage device 914 to the main storage device 913 and executes commands included in the programs, thereby causing the computer 910 to function as parts that the information processing device 1 and schedule server 80 have. Note that it is sufficient for the storage medium of the auxiliary storage device 914 that records information such as programs and so forth to be computer-readable “non-transitory tangible media”, and may be, for example, a tape, disk/disc, card, semiconductor memory, programmable logical circuit, or the like. Also, the main storage device 913 may be omitted if the computer is capable of executing programs stored in a recording medium without loading to the main storage device 913. One each or multiple of each may be provided of the above devices (computing device 912, main storage device 913, auxiliary storage device 914, input/output interface 915, communication interface 916, input device 920, and output device 930).

The aforementioned programs may be obtained from outside of the computer 910, and in such a case, may be obtained via any transmission medium (communication network, broadcast waves, etc.). The present disclosure may be realized in the form of data signals embedded in carrier waves realized by electronic transmission of the above programs.

### Summarization

A television receiver (50) according to Aspect 1 of the present disclosure is a television receiver (50) including a receiver communication unit (55), a display unit (65), and a receiver control unit (60). The receiver control unit (60) is configured to obtain public recommendation information that is obtained without referencing information of a user or a group including the user via the receiver communication unit (55), cause public recommendation information to be displayed on the display unit (65), and further cause private recommendation information that is obtained by referencing information of an individual user or a group including the user to be displayed on the display unit (65), at a certain timing.

According to the above configuration, the user is presented with public recommendation information and private recommendation information, without having to perform troublesome operations.

In Aspect 2 of the present disclosure, the television receiver (50) according to the above Aspect 1 may be configured where the receiver control unit (60) makes such an output as to cause, at the certain timing, multiple cards including the public recommendation information and at least one card including the private recommendation information to be displayed at display positions in accordance with degrees of priority correlated with respective cards, and a card including the private recommendation information to be displayed at a display position with a higher ranking of priority than multiple cards including the public recommendation information.

According to the above configuration, the user is presented with public recommendation information and private recommendation information, without having to perform troublesome operations, and also cards including the private recommendation information are displayed at display positions with a higher ranking of priority. Accordingly, information that is useful to the user is displayed in a manner that is readily comprehended by the user.

In Aspect 3 of the present disclosure, the television receiver (50) according to the above Aspect 1 or 2 may be configured where the receiver control unit (60) obtains, via the receiver communication unit (55), a group of data not including the private recommendation information and including period information indicating a display period for displaying the private recommendation information, and the receiver control unit (60) causes the private recommendation information to be displayed at at least one timing of a display period specified by the period information.

According to the above configuration, the user is presented with public recommendation information and private recommendation information, without having to perform troublesome operations.

In Aspect 4 of the present disclosure, the television receiver (50) according to any one of the above Aspects 1 to 3 may be configured where the receiver control unit (60) obtains information of a user or a group including the user from another application running on the television receiver (50), and references the information of the user or group including the user to generate the private recommendation information.

According to the above configuration, private information of a user or a group including the user is obtained from another application, such as a schedule application or the like, running on the television receiver (50), and private recommendation information is generated, so the user is presented with public recommendation information and private recommendation information, without having to perform troublesome operations.

An information processing system according to Aspect 5 of the present disclosure is an information processing system including a television receiver (50) and an information processing device (1). The information processing device (1) transmits public recommendation information that is obtained without referencing information of a user or a group including the user to the television receiver (50). The television receiver (50) includes a receiver communication unit (55), a display unit (65), and a receiver control unit (60). The receiver control unit (60) is configured to obtain public recommendation information via the receiver communication unit (55), cause public recommendation information to be displayed on the display unit (65), and further cause private recommendation information that is obtained by referencing information of a user or a group including the user to be displayed on the display unit (65), at a certain timing.

According to the above configuration, the user is presented with public recommendation information and private recommendation information, without having to perform troublesome operations.

A display method according to Aspect 6 of the present disclosure includes a transmission step of transmitting public recommendation information that is obtained without referencing information of a user or a group including the user, an obtaining step of obtaining public recommendation information at a television receiver (50), and a display step of causing public recommendation information obtained in the obtaining step to be displayed on a display unit (65). In the display step, private recommendation information that is obtained by referencing information of a user or a group including the user is further caused to be displayed on the display unit (65), at a certain timing.

According to the above configuration, the user is presented with public recommendation information and private recommendation information, without having to perform troublesome operations.

An information processing system according to Aspect 7 of the present disclosure is an information processing system including a television receiver (50), an information processing device (1), and a schedule server (80). The information processing device (1) transmits public recommendation information that is obtained without referencing private information of an individual or a group, and a group of data not including private recommendation information that is obtained by referencing private information of individual or a group and including period information indicating a display period for displaying private recommendation information, to the television receiver (50). The schedule server (80) transmits private information of an individual or group to the television receiver (50). The television receiver (50) includes a receiver communication unit (55), a display unit (65), and a receiver control unit (60). The receiver control unit (60) obtains the public recommendation information, the group of data, and the private information of the individual or group via the receiver communication unit (55), the public recommendation information is caused to be displayed on the display unit (65), and further private recommendation information that is obtained by referencing private information of an individual or a group is caused to be displayed on the display unit (65) at at least one timing of a display period specified by the period information included in the group of data.

According to the above configuration, the user is presented with public recommendation information and private recommendation information, without having to perform troublesome operations.

The receiver control unit of the television receiver and information processing device according to aspects of the present disclosure may be realized by a computer, and in this case, a control program for the television receiver and a control program for the information processing device that cause the receiver control unit and information processing device to be realized at the computer by causing the computer to operate as components (software elements) of the receiver control unit and information processing device, and a computer-readable recording medium in which these control programs are recorded, are encompassed by the scope of the present disclosure.

The present disclosure is not restricted to the above-described embodiments. Various types of modifications may be made within the scope set forth in the Claims, and embodiments obtained by appropriately combining technical measures disclosed in different embodiments also are included in the technical scope of the present disclosure. Further, new technical features may be formed by combining technical measures disclosed in the embodiments.

The present disclosure contains subject matter related to that disclosed in Japanese Priority Patent Application JP 2018-179161 filed in the Japan Patent Office on Sep. 25, 2018, the entire contents of which are hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A terminal device, comprising:
a communication unit; and
a control unit, wherein
the control unit is configured to
obtain, via the communication unit, public recommendation information that is obtained without referencing private information of an individual or a group,
output the public recommendation information for display, and
further output, at a certain timing, private recommendation information that is obtained by referencing private information of an individual or a group.

2. The terminal device according to claim 1, wherein
the control unit makes such an output as to cause, at the certain timing,
a plurality of cards including the public recommendation information and at least one card including the private recommendation information to be displayed at display positions in accordance with degrees of priority correlated with respective cards, and
a card including the private recommendation information to be displayed at a display position with a higher ranking of priority than multiple cards including the public recommendation information.

3. The terminal device according to claim 1, wherein
the control unit
obtains, via the communication unit, a group of data not including the private recommendation information and including period information indicating a display period for displaying the private recommendation information, and
outputs the private recommendation information to be displayed at at least one timing within a display period specified by the period information.

4. The terminal device according to claim 1, wherein
the control unit
obtains the private information of the individual or group from another application executed at the terminal device, and
generates the private recommendation information by referencing the private information of the individual or group.

5. An information processing system, comprising:
a terminal device; and
a server, wherein
the server
transmits public recommendation information, which is obtained without referencing private information of an individual or group, to the terminal device, and
the terminal device includes a terminal communication unit and a terminal control unit,
the terminal control unit
obtaining the public recommendation information via the terminal communication unit,
outputting the public recommendation information for display, and
further outputting private recommendation information that is obtained by referencing private information of an individual or a group, at a certain timing.

6. A method, comprising:
transmitting public recommendation information that is obtained without referencing private information of an individual or a group;
obtaining the public recommendation information at a terminal device; and
outputting the public recommendation information obtained in the obtaining, for display, wherein
in the outputting,
private recommendation information that is obtained by referencing private information of an individual or a group is further output at a certain timing.

7. An information processing system, comprising:
a terminal device;
a first server; and
a second server, wherein
the first server transmits, to the terminal device,
public recommendation information that is obtained without referencing private information of an individual or group and
a group of data not including private recommendation information that is obtained by referencing private information of an individual or group and including period information indicating a display period for displaying the private recommendation information,
the second server transmits, to the terminal device,
private information of an individual or group, and
the terminal device includes a terminal communication unit and a terminal control unit,
the terminal control unit
obtaining, via the terminal communication unit, the public recommendation information, the group of data, and the private information of the individual or group,
outputting the public recommendation information for display, and
further outputting private recommendation information that is obtained by referencing private information of an individual or a group, at at least one timing within a display period specified by the period information included in the group of data.

8. A method, comprising:
first transmitting, of public recommendation information that is obtained without referencing private information of an individual or group;
second transmitting, of a group of data not including private recommendation information that is obtained by referencing private information of an individual or group and including period information indicating a display period for displaying the private recommendation information;
obtaining, at a terminal device, the public recommendation information and the group of data; and
outputting the public recommendation information obtained in the obtaining, for display, wherein
in the outputting,
the private recommendation information is further output, at at least one timing within a display period specified by the period information included in the group of data.
